(19) United States
(12) Patent Application Publication
(10) Pub. No.: US 2024/0075251 A1
(43) Pub. Date: Mar. 7, 2024

(54) Instrument Delivery Device with Curved Nested Housing

(71) Applicant: Becton, Dickinson and Company, Franklin Lakes, NJ (US)

(72) Inventors: Weston F. Harding, Lehi, UT (US); Curtis H. Blanchard, Herriman, UT (US)

(21) Appl. No.: 17/901,026

(22) Filed: Sep. 1, 2022

(51) Int. Cl. A61M 25/06

(52) U.S. Cl. CPC A61M 25/0606; A61M 25/0662; A61M 2025/0681

(57) ABSTRACT

A medical device including an instrument, and an introducer configured to moveably receive the instrument and having an outer housing defining a curved passageway and an inner housing slidably received within the outer housing. The introducer couples to an intravenous line. The inner housing is movable relative to the outer housing and moves the instrument between a first position, in which the instrument is disposed within the outer housing, and a second position, in which the distal end of the instrument is disposed beyond the distal end of the outer housing and a portion of the instrument is disposed within the intravenous line. The curvature of the passageway of the outer housing may follow an arc of a circle or be coiled. The inner housing may be straight, curved, or coiled. Also, a system including a catheter assembly comprising a catheter adapter and a catheter, and the medical device.

### BACKGROUND OF THE INVENTION

### Field of the Invention

The present disclosure relates to a medical device for use with catheters and, more specifically, to an instrument delivery device to improve the delivery of instruments into the catheter or through the intravenous (IV) catheter and into the vasculature.

### Description of Related Art

Instrument delivery devices, including those used for collecting blood, when used with indwelling catheters, can include displaceable instruments that are advanced into and/or beyond the tip of the indwelling catheter. Often, when the displaceable instrument is advanced, it can encounter an obstruction, resulting in deflection of the instrument. Examples of obstructions include the friction of the seal within the delivery device, a torturous path within an integrated catheter, pinching of the catheter tubing as the catheter extends into the skin, thrombus, fibrin, and valves. Further, delivery of instruments can be complicated by the interchange of the instruments, and the necessary length needed for deployment of such instruments. Accordingly, a need exists in the art for instrument delivery devices that allow for robust delivery and performance of the instrument that is being introduced into the catheter and/or vasculature.

### SUMMARY OF THE INVENTION

The present invention is directed to a medical device comprising an instrument having a proximal end and a distal end, and an introducer configured to moveably receive the instrument. The introducer has an outer housing having a proximal end, a distal end, and a sidewall therebetween defining a passageway, wherein the passageway is curved, and an inner housing having a proximal end, a distal end, and a sidewall therebetween defining an inner volume. The passageway of the outer housing is curved, and the outer housing may also be curved. The inner housing is slidably received within the outer housing. A distal end of the introducer is configured to couple the introducer to an intravenous line, and the inner housing is movable relative to the outer housing and moves the instrument between a first position, in which the instrument is disposed within the outer housing, and a second position, in which the distal end of the instrument is disposed beyond the distal end of the outer housing such that at least a portion of the instrument is disposed within the intravenous line when the introducer is coupled to the intravenous line.

A curvature of the passageway of the outer housing follows an arc of a circle. A curvature of the outer housing may also follow an arc of a circle. The inner housing may be straight or may be curved along an arc of a circle. The inner housing may extend along an arc of the circle corresponding to the arc of the circle along which the outer housing extends. The inner housing and the outer housing may extend along substantially equal arcs of the circle. When the inner housing is in the first position, the combined inner housing and outer housing may extend around a partial circle, may extend around a full circle, or may extend around more than a full circle.

The passageway of the outer housing may be coiled having at least one rotation extending around a full circle.

The outer housing may be tubular and coiled with concentric rotations having a set shape that is provided by a material of the outer housing. Concentric rotations of the outer housing may be attached to one another along all or a portion of a contact area between the concentric rotations. The outer housing may be tubular and concentric rotations of the outer housing may be attached to one another by at least one connector.

The outer housing may be a single or multiple piece housing that defines a coil-shaped passageway. An exterior of the outer housing may have a shape other than a coiled shape and the passageway defined within the outer housing is coil shaped. The outer housing may be an assembly of two or more parts that are connected in a clam shell configuration.

The inner housing may be straight. The inner housing may be coiled, have a larger diameter than a diameter of the outer housing, and surround the outer housing, or the outer housing may be coiled and the inner housing may be coiled, and the inner housing may have a smaller diameter than a diameter of the outer housing and be positioned within a center of the outer housing. The inner housing may be coiled and be positioned adjacent to the outer housing. The inner housing may comprise any number of full and/or partial rotations.

A length of the inner housing may be approximately equal to a length of the outer housing.

The present invention is further directed to a system comprising a catheter assembly and the medical device described above. The catheter assembly comprises a catheter adapter and a catheter extending distally from the catheter adapter. The catheter adapter comprises a distal end, a proximal end, a lumen extending between the distal end and the proximal end, and a side port arranged between the distal end and the proximal end, the side port in fluid communication with the lumen. The medical device is attached to the side port and the catheter is in fluid communication with the lumen.

The medical device may be coupled to the side port by a fluid conduit having a proximal end coupled to the side port and a distal end coupled to the medical device.

The instrument may be one or more of a catheter, a guidewire, an obturator, a wire, electrical wiring, a probe, a light pipe, and a sensor.

### BRIEF DESCRIPTION OF THE DRAWINGS

FIGS. 1A-1B, 2A-2B, 3A,-3B and 4A-4B show top views of instrument delivery devices and systems according to non-limiting embodiments described herein;

FIGS. 5A-5C show top schematic views of the outer housing and the inner housing of instrument delivery devices according to non-limiting embodiments described herein;

FIG. 6 shows a top view of an instrument delivery device and system according to non-limiting embodiments described herein;

FIG. 7 shows a partial top view of an instrument delivery device and system according to non-limiting embodiments described herein;

FIG. 8 shows a partial top view of an instrument delivery device and system according to non-limiting embodiments described herein;

FIG. 9 shows a perspective view of an instrument delivery device according to non-limiting embodiments described herein;

FIG. 10 shows a partial top view of an instrument delivery device and system according to non-limiting embodiments described herein;

FIGS. 11A-11C show partial views of an instrument delivery device according to non-limiting embodiments described herein;

FIGS. 12A-12B show partial views of an instrument delivery device according to non-limiting embodiments described herein; and

FIG. 13 shows a perspective view of an instrument delivery device according to non-limiting embodiments described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, equivalents, variations, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value equal to or less than 10.

Provided herein are devices and systems for delivering instruments through indwelling catheters, such as peripheral intravenous catheters (PIVCs). While certain devices (e.g., blood draw devices) are discussed below in terms of devices that may be used with PIVCs, and exemplified in the attached drawings, those of skill will appreciate that any number of different devices for introducing an instrument, including instruments ranging from tubes, probes, sensors (e.g., pressure sensors, pH sensors, lactate sensors, glucose sensors, and the like), wiring, fiber optics, guidewires, etc., may be used within the scope of the present disclosure.

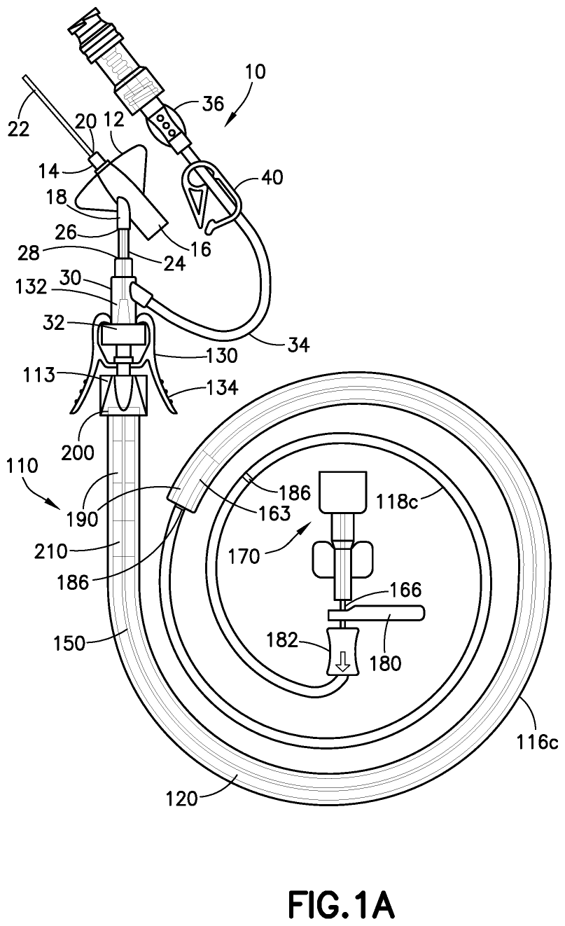

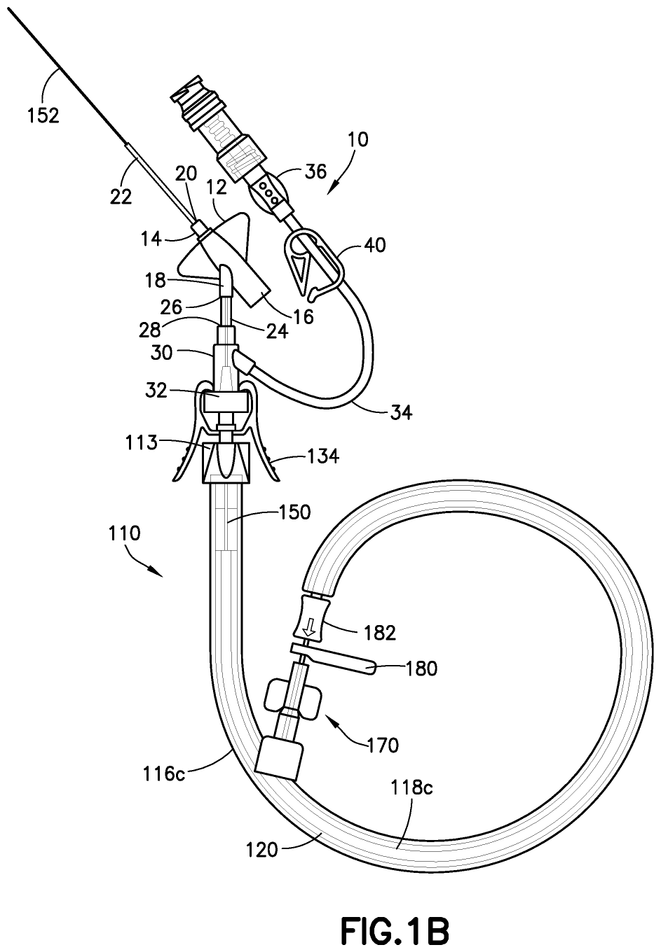

FIGS. 1A-4B show non-limiting embodiments of a system including a catheter assembly 10 and instrument delivery device 110. Suitable catheter assemblies for use with the instrument delivery devices 110 described herein are commercially available, for example, from Becton, Dickinson and Company under the trade name Nexiva. The catheter assembly 10 may include a catheter adapter 12, which may have a distal end 14 and a proximal end 16. In some embodiments, the catheter adapter 12 may include one or more additional ports 18. In some embodiments, the additional port 18 may be disposed between the distal end 14 and the proximal end 16 of the catheter adapter 12. In some embodiments, more than one port 18 may be disposed between the distal end 14 and the proximal end 16 of the catheter adapter 12. In some embodiments, the additional port 18 may be disposed at the proximal end 16 of the catheter adapter 12. In some embodiments, the catheter adapter 12 may include a first lumen 20 extending through the distal end 14 and the proximal end 16 of the catheter adapter 12. The first lumen 20 may be sealed at the proximal end 16 of catheter adapter 12.

In some non-limiting embodiments, the catheter assembly 10 may include a catheter 22 extending from the distal end 14 of the catheter adapter 12. The catheter 22 may be any catheter suitable for accessing the vasculature of a patient, including, but not limited to, a peripheral intravenous catheter, a midline catheter, an arterial catheter, a peripherally-inserted central catheter (PICC), or a central venous catheter (CVC) access device (VAD), including integrated or non-integrated vascular access devices. The catheter 22 may be formed of any suitable material and may be of any useful length, as known to those of skill in the art. In some non-limiting embodiments, the catheter assembly 10 may include a first fluid conduit 24 extending from the port 18. The first fluid conduit 24 may be formed of any suitable material known to those of skill in the art, and may have a distal end 26 and a proximal end 28. The first fluid conduit 24 may be coupled, at the distal end 26 thereof, to the port 18. In some non-limiting embodiments, a connector 30 may be coupled to a proximal end 28 of first fluid conduit 24. The connector 30 may be a t-connector (e.g., one side port arranged at a 90 degree angle relative to a longitudinal axis of the connector 30), a y-connector (e.g., one side port arranged at a 15-165 degree angle relative to a longitudinal axis of the connector 30), or any other type of connector known in the art, and may include a second lumen therethrough, having any number of branches suitable for the type of connector 30.

The catheter assembly 10 may include a needleless access connector 32 and/or a second fluid conduit 34. Suitable needleless access connectors 32 can include any split-septum connector and/or those with direct fluid path access. Needleless access connectors 32 are known to those of skill in the art and are commercially available from, for example, Becton, Dickinson and Company under the trade names Q-SYTE, and SMARTSITE. While the non-limiting embodiments of FIGS. 1A-4B show needleless access connectors 32 arranged at connector 30, those of skill in the art will appreciate that suitable needleless access connectors 32 may also be arranged at the luer connection 36. In non-limiting embodiments, the needleless access connector 32 includes a septum (not shown), such as a self-healing septum. In non-limiting embodiments, the septum is a slit-type septum. As will be described below, the instrument delivery device 110 may be reversibly coupleable to the needleless access connector 32, and one or more portions of the instrument delivery device 110 may pierce the septum and access the patient's vasculature through the catheter 22.

In some non-limiting embodiments, the catheter assembly 10 may include an extension set (integrated into or removably coupleable to catheter adapter 12, connector 30, and/or needleless access connector 32) including a second fluid conduit, such as second fluid conduit 34. Extension sets are known to those of skill in the art and are commercially available from, for example, Becton, Dickinson and Company. In some non-limiting embodiments, the second fluid conduit 34 may include a luer connection 36 at an end thereof. In some non-limiting embodiments, the extension set may include a clamp 40 to allow for occlusion of second fluid conduit 34. The clamp 40 and the second fluid conduit 34 may be formed of any suitable materials known to those of skill in the art. In non-limiting embodiments, the second lumen (e.g., within connector 30) has an inner diameter that is substantially equivalent to an inner diameter of the first fluid conduit 24 and/or the second fluid conduit 34.

With reference to FIGS. 1A-4B, the instrument delivery device 110 includes an outer housing 116 having a proximal end, a distal end, and a sidewall defining a passageway 120, and an inner housing 118 having a proximal end, a distal end, and a sidewall defining an inner volume slidably received within the outer housing 116. In non-limiting embodiments, the inner housing 118 and the outer housing 116 are in a telescoping relationship, such that the inner housing 118 may be slidably received entirely, or almost entirely, within the outer housing 116. In non-limiting embodiments, the inner housing 118 may have a variable diameter along its length, for example, a distal end of the inner housing 118 may be of a larger diameter than other portions of the inner housing 118.

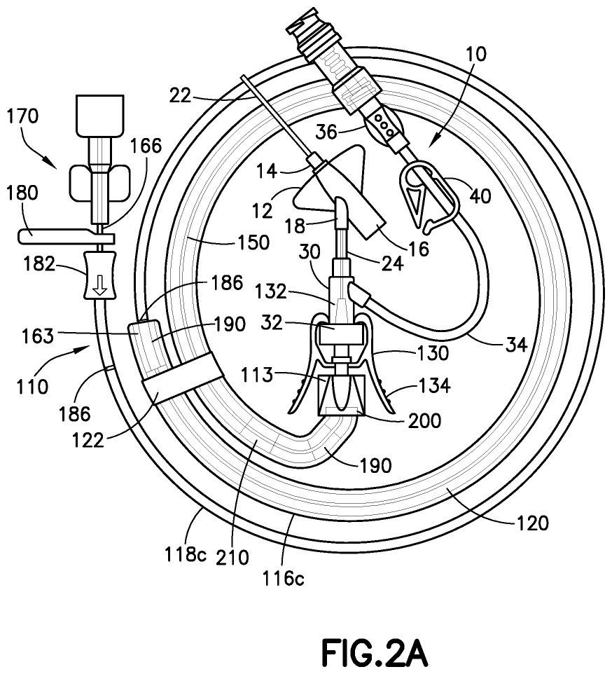

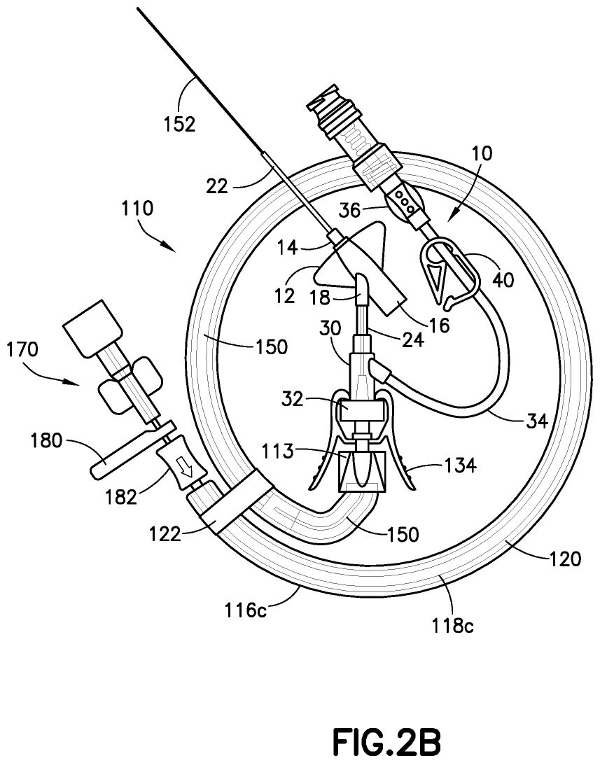

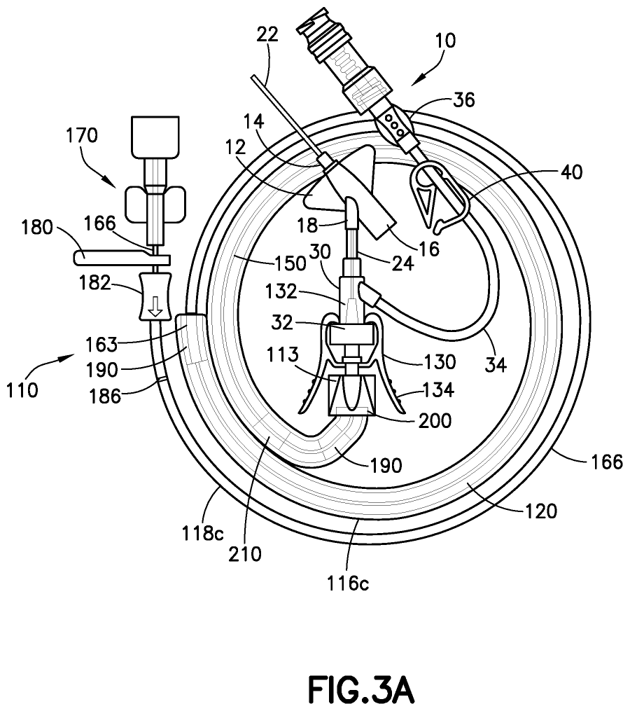

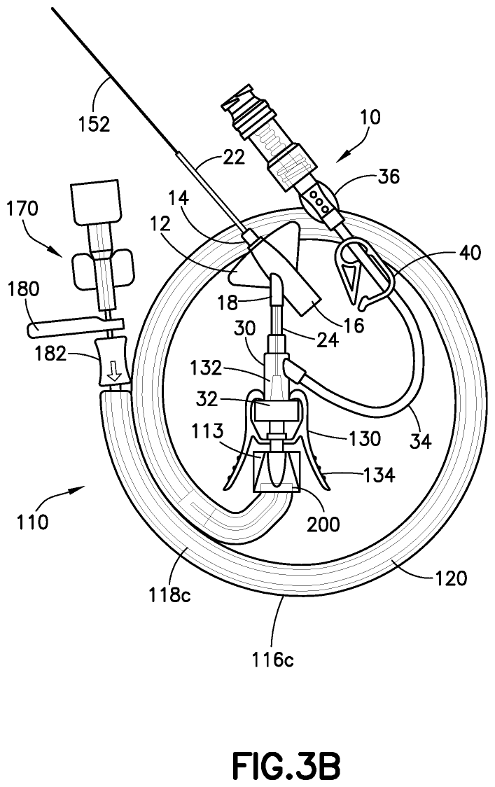

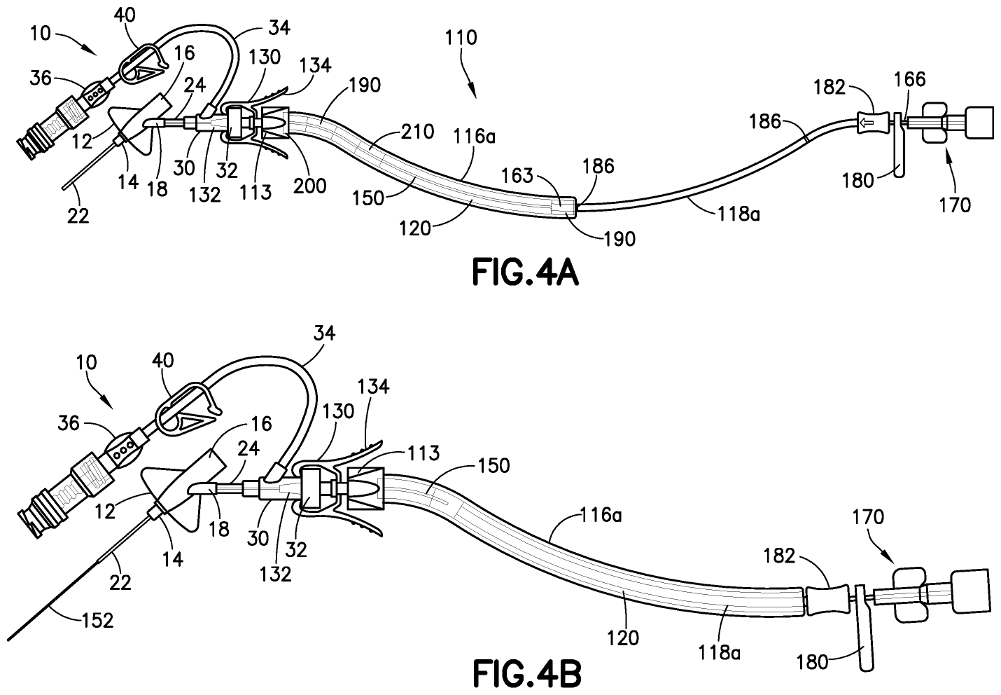

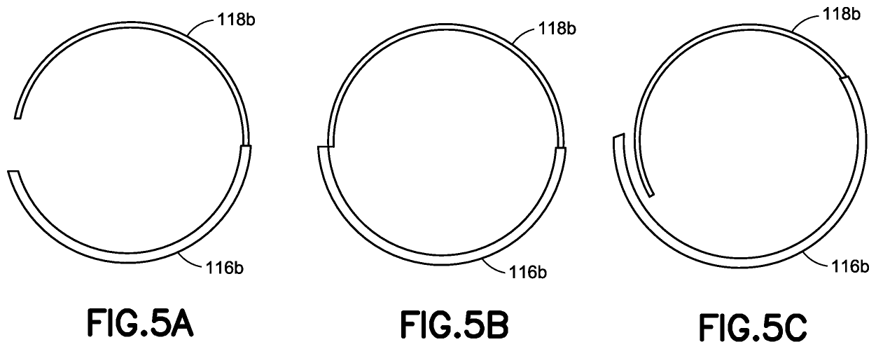

The outer housing 116 defines a curved passageway 120 through which the inner housing 118 is received. In one non-limiting embodiment, the curvature of the passageway 120 of the outer housing 116 may follow at least an arc of a circle. The curvature of the passageway 120 of the outer housing 116a may have a curved arch shape (arc of a large radius circle) as shown in FIGS. 4A and 4B, the outer housing 116b may extend around a significant portion of a circle (FIGS. 5A and 5B) (arc of a small radius circle), or the outer housing 116c may take the form of a coil having at least one rotation extending around a full circle (FIGS. 1A-3B) The coiled outer housing 116c may comprise any number of full and/or partial rotations, for example, a coil having at least one full circular rotation (FIGS. 1A and 1B), or a coil having more than one full circular rotation (FIGS. 2A-3B).

In one non-limiting embodiment (FIGS. 1A and 1B), the outer housing 116c may be tubular and may be coiled with concentric rotations of the outer housing 116c coiled with a set shape that is provided by the material of the outer housing 116c. Concentric rotations of the outer housing 116c may be separate from one another (FIGS. 1A and 1B) or may be attached to one another along all or a portion of the contact area between the concentric rotations.

In another non-limiting embodiment (FIGS. 2A and 2B), the outer housing 116a may be tubular with concentric rotations of the outer housing 116c attached to one another by at least one connector 122.

In yet a further non-limiting embodiment (FIGS. 3A and 3B), the outer housing 116c may be a single or multiple piece housing that defines a coil-shaped passageway 120 through which the inner housing 118 is received. While the exterior of the outer housing 116c may have a coiled shape as shown in FIGS. 3A and 3B, the exterior of the outer housing 116c may have any shape as long as the passageway 120 defined within the outer housing 116c is coil shaped. Such an outer housing 116c may be an assembly of two or more parts that are connected in a clam shell configuration.

The outer housing 116 may be rigid, semi-flexible, or flexible. For example, the outer housing 116 may be made from any suitable material including plastics. For example, the outer housing 116 may be made from polyurethane, polycarbonate, and/or polypropylene. The material of the outer housing 116 may be clear, i.e., transparent or semi-transparent.

The inner housing may be straight (not shown), or the inner housing 118 may be curved along at least an arc of a circle (FIGS. 1A-4B). In a non-limiting embodiment, the inner housing 118a, 118b may be a rigid or semi-rigid tube with the inner housing extending along an arc of the circle corresponding to the arc of the circle along which the outer housing 116a, 116b extends (FIGS. 4A-5C). The inner housing 118a, 118b and the outer housing 116a, 116b may extend along substantially equal arcs of the circle. When the inner housing 118b is in a first position extending from the outer housing 116b, the combined inner housing 118b and outer housing 116b may extend around a partial circle (FIGS. 4A, 4B, and 5A), a full circle (FIG. 5B) or more than a full circle (FIG. 5C).

In another non-limiting embodiment, the inner housing 118c may be coiled with concentric rotations of the inner housing 118c in a set shape that is provided by the material of the inner housing 118c (FIGS. 1A-3B). Alternatively, the inner housing 118 may be flexible so that it bends as the inner housing 118 enters the outer housing 116.

In further non-limiting embodiments, the inner housing 118c may be coiled, may have a larger diameter than a diameter of the coiled outer housing 116c, and may surround the coiled outer housing 116c (FIGS. 2A-3B). The inner housing 118c may be coiled, may have a smaller diameter than a diameter of the coiled outer housing 116c, and may be positioned within the center of the coiled outer housing 116c (FIGS. 1A and 1B). The inner housing 118c may be coiled, have a diameter the same as or different from a diameter of the coiled outer housing 116c and be positioned adjacent to the coiled outer housing 116c. The coiled inner housing 118c may comprise any number of full and/or partial rotations, for example, a coil having at least one full circular rotation, or a coil having more than one full circular rotation.

The inner housing 118 may be made of any suitable material that minimizes potential buckling of the inner housing 118 including, for example, polymeric and elastomeric materials, such as polyethylene, polypropylene, nylon, polyurethane, and the like, and metallic materials. Those of skill will appreciate that various materials may be suitable, so long as they minimize potential buckling of the inner housing 118.

In a non-limiting embodiment, a length of the inner housing 118 is approximately equal to a length of the outer housing 116.

In non-limiting embodiments, the instrument delivery device 110 may include a lock 130 at a distal end for attachment of the instrument delivery device 110 to a catheter 22 or a catheter adapter 12. In non-limiting embodiments, the lock 130 may be a proboscis 132 coupled with locking arms 134. The proboscis 132 is configured to provide a fluid-tight seal with the catheter assembly 10, for example, by having a diameter that is closely matched to a diameter of the catheter adapter 12 and/or the needleless access connector 32, thereby limiting the ability of fluid to escape between an outer wall of the proboscis 132 and an inner wall of the catheter adapter 12 and/or the needleless access connector 32. While the lock 130 is exemplified as a proboscis 132 and locking arms 134 in FIGS. 1A-4B and FIGS. 6-8, those of skill will appreciate that any type of suitable connection may be used to secure the instrument delivery device 110 to an indwelling catheter 22 or a catheter assembly 10, including a luer connection, a clip, a blunt plastic cannula, a blunt metal cannule, a hybrid luer (e.g., with a cannula), a friction fit, and the like.

The instrument delivery device 110 further includes an instrument 150, exemplified in the drawings as a catheter or fluid conduit, having a proximal end and a distal end 152. As described previously and as will be appreciated by those of skill in the art, the instrument may be any medical instrument that can be delivered through the catheter assembly 10 to a patient's vasculature, for example, one or more of a fluid conduit, a catheter, a guidewire, an obturator, a wire, electrical wiring, a probe, a light pipe, and a sensor. Instruments useful with the instrument delivery device 110 described herein may be formed of any useful material, for example, the instrument 150 may be a fluid conduit formed of a polymer, such as a polyimide-containing material, a nylon, a polyurethane, or another suitable polymeric material.

The instrument 150 is slidably received within the outer housing 116, and may be advanced and/or retracted relative to outer housing 116 by displacement of the inner housing 118 relative to outer housing 116. FIGS. 1A, 2A, 3A, and 4A show the instrument delivery device 110 in a first state, where the distal end 152 of the instrument 150 is in a first position, received within the outer housing 116 and/or the lock 130, and the inner housing 118 is in a first position, extending proximally from the outer housing 116. FIGS. 1B, 2B, 3B, and 4B show the instrument delivery device 110 in a second state, where the distal end 152 of the instrument 150 is in a second position, extending distally from the outer housing 116 and/or the lock 130, and the inner housing 118 is in a second position, received within the outer housing 116.

The length of the outer housing 116 and inner housing 118 of the instrument delivery device 110 in the first position in which the inner housing 118 extends from the outer housing 116 must be substantially longer than the instrument 150 being inserted into the vasculature, and is generally twice the length of the instrument 150. Therefore, when a straight outer housing and a straight inner housing are provided, the instrument delivery device 110 can be cumbersome and difficult to use. In the inventive instrument delivery device 110, the curved outer housing 116 and inner housing 118, especially the coiled outer housing 116c and inner housing 118c, reduce the overall length of the instrument delivery device 110 making the instrument delivery device 110 more compact and easier to use, especially when the outer housing 116 and/or the inner housing 118 are coiled.

Further, the curved instrument delivery device 110 having a bow-shaped outer housing 116a and inner housing 118a allows the user to preselect the orientation of the curve to enable better access to the intravenous catheter by allowing the instrument delivery device 110 to curve around the arm of the patient, the bed of the patient, or other obstructions. For example, if the instrument delivery device 110 were being utilized on the left arm of the patient, the selected orientation of the curve of the instrument delivery device 110 may be oriented differently than when the instrument delivery device 110 is being utilized on the right arm of the patient. Or, if the arm of the patient were in a location in a bed with guard rails, the curved instrument delivery device 110 allows adjustments that make the instrument delivery device 110 more ergonomic to use. The pre-curved instrument 150 in the curved housing 116a, 118a also gives the user the ability to steer the instrument 150 as it passes through the catheter adapter 12 and the vasculature. This can be accomplished by rotating the instrument delivery device 110 to orient the curvature to place the instrument 150 in the desired advancement direction.

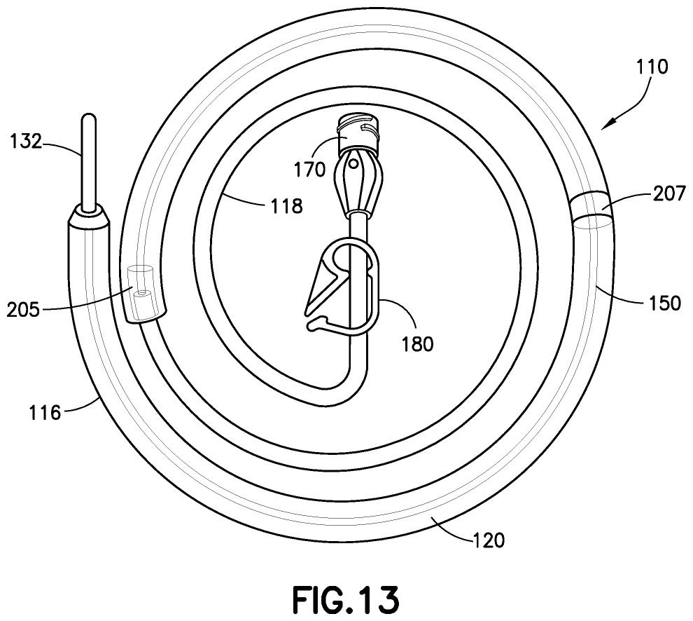

In non-limiting embodiments, the inner housing 118 is coupled to or otherwise interacts with the instrument 150, such that, as the inner housing 118 is advanced distally to the second position, for example, by way of a user grasping the grip 182 and applying a distally-directed force to the inner housing 118, the instrument 150 is moved to a second position, where a distal end 152 of the instrument 150 extends beyond the outer housing 116, the lock 130 (if present), and/or the catheter 22. The grip 182 may be arranged at a proximal end of the inner housing 118, and may be arranged at or near a clamp 180 suitable for occluding fluid flow through the inner housing 118 and/or the instrument 150, as will be described in greater detail below. The grip 182 may be formed of an ergonomic material to provide comfort while a user grips the instrument delivery device 110, and may include features to, for example, increase grip and prevent slippage while the inner housing 118 is being advanced/retracted. The clamp 180 may be a slide clamp, for example, as shown in FIGS. 1A-4B, or may be a pinch clamp, as shown in FIG. 13.

As described above, a distal portion of the inner housing 118 may be of a larger diameter than other portions of the inner housing 118, such that, as the inner housing 118 is retracted, one or more features on the outer housing 116 interact with the enlarged portion of the inner housing 118 to prevent the inner housing 118 from being completely pulled from the outer housing 116. The enlarged distal portion of the inner housing 118 may include vents for allowing air to pass therethrough, thereby reducing the force needed to advance/retract the inner housing 118 within the outer housing 116, and, as described below, lubricant 190 may be applied to the enlarged distal portion of the inner housing 118 to reduce friction between the inner housing 118 and the outer housing 116.

In non-limiting embodiments, any portion of the instrument delivery device 110 may be flexible to allow pivoting and/or rotational freedom when using the instrument delivery device 110.

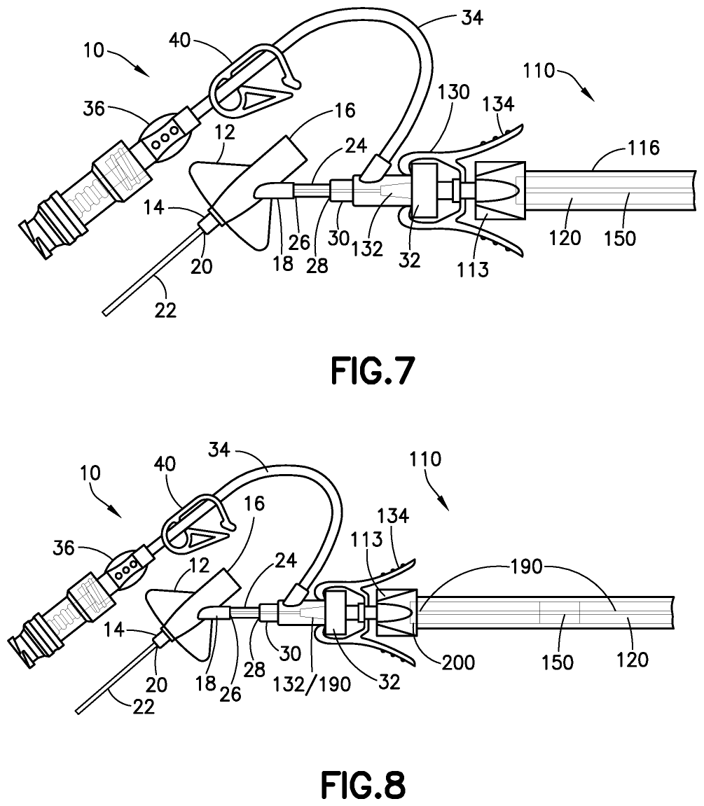

As will be described below, and as shown in the accompanying drawings, the instrument delivery device 110 may include any number of features to provide a more robust system for accessing a patient's vasculature. With continuing reference to FIGS. 1A and 1B, as well as FIG. 7, in non-limiting embodiments, a joint 113 may be included at a distal end of the outer housing 116, for example, between the outer housing 116 and the lock 130. Suitable joints 113 may include pivoting joints and rotating joints. Such joints 113 may increase the usability of the instrument delivery device 110, for example, by allowing for greater access in terms of distance from the patient's skin, as well as securement of the indwelling catheter by avoiding excessive manipulation of the catheter assembly 10 during instrument delivery and medical procedures associated therewith.

In non-limiting embodiments, the proboscis 132 of the lock 130 is formed of a flexible material to allow similar pivoting and/or rotational freedom to avoid excessive manipulation of the catheter assembly 10.

The inner housing 118 may include, at a proximal end thereof, a connector 170 to allow various medical devices to be attached to the inner housing 118, for example, to provide an instrument 150 that is to be advanced into the patient's vasculature, to inject a composition into the vasculature, and/or to receive fluid withdrawn from the vasculature. Suitable connectors 170 include luer connectors, luer lock access devices, needleless access connectors, and the like, known to those of skill in the art. The connectors 170 may also include electrical connectors, optical connectors, or a combination thereof.

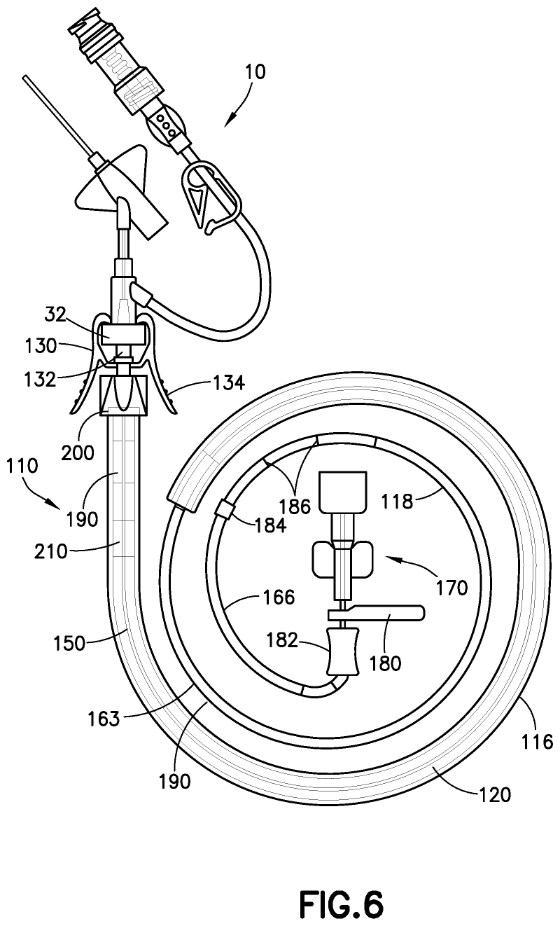

With continuing reference to FIGS. 1A, 2A, 3A, and 4A, as well as FIG. 6, in non-limiting embodiments, the inner housing 118 may include one or more indicia 184, 186 arranged on an outer surface thereof. Suitable indicia may be visual and/or tactile, and may be provided to, for example, indicate instrument length, and/or instrument positioning relative to the indwelling catheter 22. In non-limiting embodiments, one or more indicia 186 may be provided on the inner housing 118, and the outer housing 116 may be formed of a material that is at least partially transparent to allow for visualization of the indicia 186 throughout the transition of the inner housing 118 from a first position where the inner housing 118 is substantially outside of the outer housing 116 to a second position in which the inner housing 118 is substantially within the outer housing 116. In non-limiting embodiments, a separate indicia, in the form of a tactile stop 184, may be included at one or more locations along the inner housing 118 to provide a user with a tactile indication of certain thresholds. For example, a tactile stop 184 may indicate that the instrument 150 has nearly reached its full extension, may indicate that the instrument 150 can be extended no further (for example, as shown in FIG. 1B), and/or may indicate that the instrument 150 is approaching the vasculature of the patient.

Alternatively or in addition to indicia 184, 186 provided on the inner housing 118, indicia, as described above, may be provided on the outer housing 118.

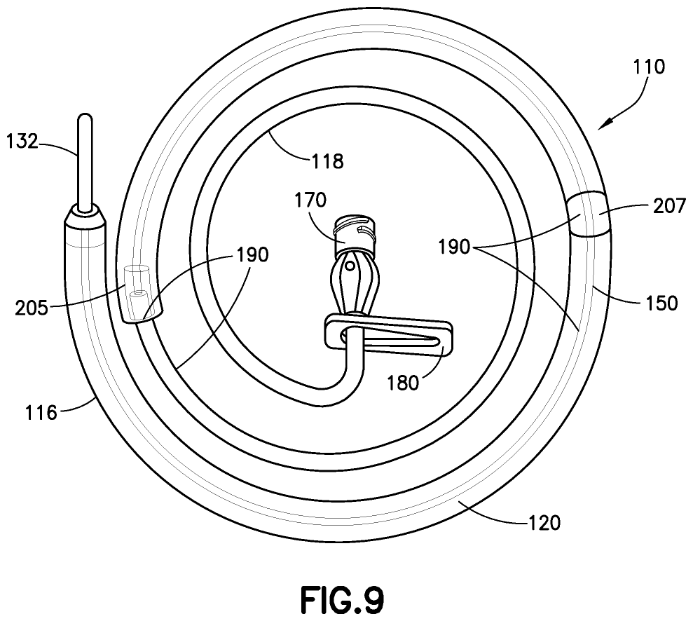

As also shown in FIG. 9, as well as FIG. 13, one or more septa 200, and one or more seals 205, 207 may be arranged about a portion of the instrument 150, and be movable with the instrument 150 through the outer housing 116. Seals 205, 207 may be arranged at a proximal end of the instrument 150 or at any suitable location along the instrument 150. The seals 205, 207 and the septa 200 may include air venting features to allow for ease of advancement and withdrawal of the instrument 150.

With continuing reference to FIGS. 1A-4B, as well as FIGS. 6, 8, and 9, in non-limiting embodiments, a lubricant 190 may be applied at one or more locations on or within one or more components of instrument delivery device 110. For example, a lubricant 190 may be applied to an outer surface of a distal portion (e.g., proboscis 132) of the outer housing 116 of the instrument delivery device 110 that is inserted into the indwelling catheter 22, such as catheter assembly 10, to decrease the force necessary to couple the devices together. The addition of a lubricant 190 on the proboscis 132 can ease connection of the instrument delivery device 110 to catheter assembly 10, despite the closeness of the respective diameters. A lubricant 190 may be applied at one or more locations within the outer housing 116, for example, on one or more outer surfaces of inner housing 118, or one or more outer surfaces of the instrument 150. Lubricant 190 may be applied to an outer surface of one or more of the seals 205, 207. Lubricant 190 may also, or alternatively, be provided about an opening through the septa 200 through which the instrument 150 may pass. Suitable lubricants are known to those of skill in the art, and may include silicone-based lubricants.

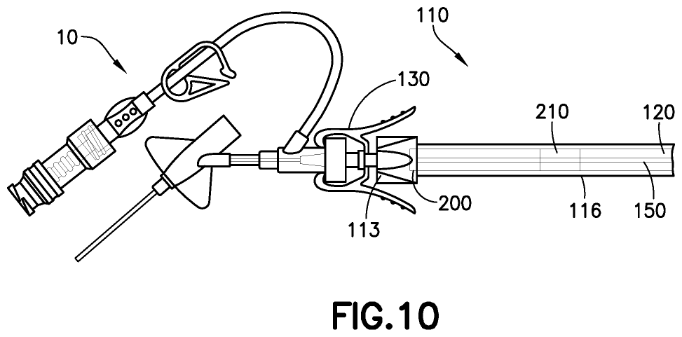

With reference to FIGS. 1A-4B, as well as FIGS. 6 and 10, in non-limiting embodiments, the instrument delivery device 110 includes one or more supports 210 arranged within the outer housing 116 to limit and/or prevent buckling of the inner housing 118 and/or the instrument 150 as the inner housing 118 and/or the instrument 150 are advanced distally through the outer housing 116. The supports 210 may include narrowed portions of the passageway 120 of the outer housing 116, one or more washers arranged about the inner housing 118 and/or the instrument 150, and/or the like, in order to reduce the effective buckling length and/or the buckling mode shape of the inner housing 118 and/or the instrument 150. Suitable supports are also described in U.S. Provisional Patent Application No. 63/273,226, filed Oct. 29, 2021, the contents of which are incorporated herein by reference in their entirety.

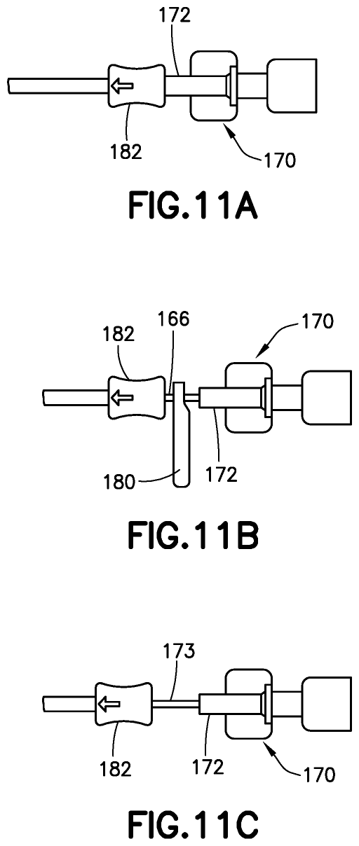

With reference to FIGS. 1A-4B, as well as FIGS. 11A-11C, in non-limiting embodiments, for example, where the instrument 150 is a fluid conduit, fluid, such as blood, may be transferred into or from the patient's vasculature in which a catheter, such as catheter 22, may be indwelling. In non-limiting embodiments, such as shown in FIGS. 1A, 2A, 3A, 4A, and 11B, the fluid conduit instrument 150 may be joined at junction 163 to a separate fluid conduit 166 that passes through the inner housing 118. The separate fluid conduit 166 may extend beyond a proximal end of the inner housing 118, for example, as shown in FIG. 11B, and may be coupled to a connector 170, optionally, through an adapter 172. In non-limiting embodiments, the fluid conduit instrument 150 is of a sufficient length that it passes directly through the inner housing 118 to couple with the connector 170, optionally, through an adapter 172. In non-limiting embodiments, for example as shown in FIGS. 11A and 11C, the inner housing 118 itself forms a fluid conduit in fluid communication with the fluid conduit instrument 150. In the embodiment of FIG. 11C, a separate fluid tube 173 is provided at the proximal end of the inner housing 118, and fluidly couples the inner housing 118 and the connector 170, optionally, through an adapter 172. In any of the described embodiments, an adapter 172 may be provided to fluidly couple the fluid conduit 166, the inner housing 118, and/or the fluid conduit instrument 150 to a connector 170, such as those described herein above.

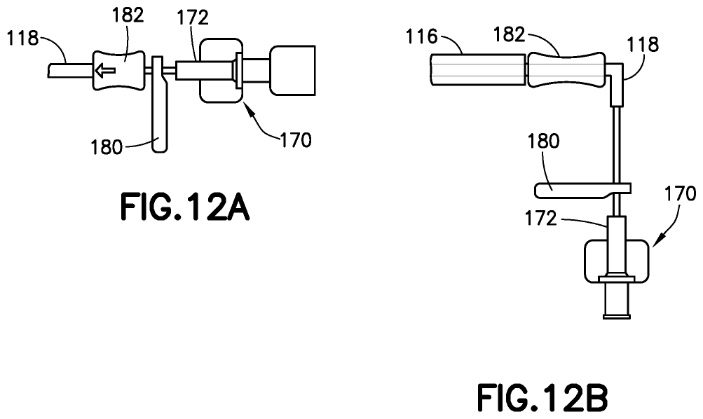

With respect to the connector 170 discussed above, turning to FIGS. 12A and 12B, various non-limiting arrangements of a connector 170 are shown. As shown in FIG. 12A, the connector 170 may be coupled to a fluid conduit (e.g., fluid conduit instrument 150 or fluid conduit 166), with a clamp 180 and a grip 182 arranged distally of the connector 170. The connector 170 may, as described above, be a luer connector, a luer lock access device, a needleless access connector, and the like known to those of skill in the art. As shown in FIG. 12B, the outer housing 118 may include an angled portion, for example, at a proximal end thereof, such that the connector 170 is arranged non-parallel to a longitudinal axis of the outer housing 116. In any embodiment, the connector 170 may be coupled to the inner housing 118 and/or the fluid conduits (e.g., fluid conduit instrument 150 or fluid conduit 166) through an adapter 172.

With further reference to the figures, in non-limiting embodiments, the inner housing 118 and the outer housing 116 may be configured, together with the clamp 180, to provide for blood withdrawal through the fluid conduit instrument 150, when the fluid conduit instrument 150 is in a second positon where a distal end 152 of the fluid conduit instrument 150 is positioned within a patient's vasculature. In non-limiting embodiments, blood can be withdrawn into the fluid conduit instrument 150 while the clamp 180 is in a first configuration, preventing fluid flow to the connector 170. With that fluid path occluded, blood may, for example, flow through one or more openings in the fluid conduit instrument 150 and/or the fluid conduit 166, filling a volume within the outer housing 116, for example, between the outer housing 116 and the inner housing 118 and/or between an outer surface of the fluid conduit instrument 150 and/or the fluid conduit 166 and an inner surface of the inner housing 118. After the volume is filled, the clamp 180 may be reoriented to a second configuration, where fluid flow to the connector 170 is not obstructed. As the volume between an outer surface of the fluid conduit instrument 150 and/or the fluid conduit 166 and an inner surface of the inner housing 118 is already filled, a fresh blood sample may be pulled through to the connector 170, and any medical device coupled thereto.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A medical device, comprising:
an instrument having a proximal end and a distal end;
an introducer configured to moveably receive the instrument and having:
an outer housing having a proximal end, a distal end, and a sidewall therebetween defining a passageway, wherein the passageway is curved; and
an inner housing having a proximal end, a distal end, and a sidewall therebetween defining an inner volume, the inner housing slidably received within the outer housing,
wherein a distal end of the introducer is configured to couple the introducer to an intravenous line, and the inner housing is movable relative to the outer housing and moves the instrument between a first position, in which the instrument is disposed within the outer housing, and a second position, in which the distal end of the instrument is disposed beyond the distal end of the outer housing such that at least a portion of the instrument is disposed within the intravenous line when the introducer is coupled to the intravenous line.

2. The medical device of claim 1, wherein the outer housing is curved.

3. The medical device of claim 1, wherein a curvature of the passageway of the outer housing follows an arc of a circle.

4. The medical device of claim 1, wherein a curvature of the outer housing follows an arc of a circle.

5. The medical device of claim 1, wherein the inner housing is straight.

6. The medical device of claim 1, wherein the inner housing is curved along an arc of a circle.

7. The medical device of claim 1, wherein the inner housing extends along an arc of the circle corresponding to an arc of the circle along which the outer housing extends.

8. The medical device of claim 1, wherein the inner housing and the outer housing extend along substantially equal arcs of the circle.

9. The medical device of claim 6, wherein, when the inner housing is in the first position, the combined inner housing and outer housing extend around a partial circle.

10. The medical device of claim 6, wherein, when the inner housing is in the first position, the combined inner housing and outer housing extend around a full circle.

11. The medical device of claim 6, wherein, when the inner housing is in the first position, the combined inner housing and outer housing extend around more than a full circle.

12. The medical device of claim 1, wherein the passageway of the outer housing is coiled having at least one rotation extending around a full circle.

13. The medical device of claim 12, wherein the outer housing is tubular and coiled with concentric rotations having a set shape that is provided by a material of the outer housing.

14. The medical device of claim 12, wherein concentric rotations of the outer housing are attached to one another along all or a portion of a contact area between the concentric rotations.

15. The medical device of claim 12, wherein the outer housing is tubular and concentric rotations of the outer housing are attached to one another by at least one connector.

16. The medical device of claim 11, wherein the outer housing is a single or multiple piece housing that defines a coil-shaped passageway.

17. The medical device of claim 16, wherein an exterior of the outer housing has a shape other than a coiled shape and the passageway defined within the outer housing is coil shaped.

18. The medical device of claim 16, wherein the outer housing is an assembly of two or more parts that are connected in a clam shell configuration.

19. The medical device of claim 11, wherein the inner housing is straight.

20. The medical device of claim 11, wherein the inner housing is coiled, has a larger diameter than a diameter of the outer housing, and surrounds the outer housing.

21. The medical device of claim 11, wherein the outer housing is coiled and the inner housing is coiled, and the inner housing has a smaller diameter than a diameter of the outer housing and is positioned within a center of the outer housing.

22. The medical device of claim 11, wherein the inner housing is coiled and is positioned adjacent to the outer housing.

23. The medical device of claim 20, wherein the inner housing comprises any number of full and/or partial rotations.

24. The medical device of claim 1, wherein a length of the inner housing is approximately equal to a length of the outer housing.

25. A system, comprising:
a catheter assembly comprising:
a catheter adapter comprising
a distal end;
a proximal end;
a lumen extending between the distal end and the proximal end; and
a side port arranged between the distal end and the proximal end, the side port in fluid communication with the lumen; and
a catheter extending distally from the catheter adapter and in fluid communication with the lumen; and
the medical device of claim 1, coupled to the side port.

26. The system of claim 25, wherein the medical device is coupled to the side port by a fluid conduit having a proximal end coupled to the side port and a distal end coupled to the medical device.

27. The system of claim 25, wherein the instrument is one or more of a catheter, a guidewire, an obturator, a wire, electrical wiring, a probe, a light pipe, and a sensor.
